(19) United States
(12) Patent Application Publication
(10) Pub. No.: US 2024/0148930 A1
(43) Pub. Date: May 9, 2024

(54) STERILIZING PANEL LAMP

(71) Applicant: Shenzhen Guanke Technologies Co., Ltd., Shenzhen (CN)

(72) Inventors: Qing LAN, Shenzhen (CN); Ligen LIU, Shenzhen (CN); Penghui LIU, Shenzhen (CN); Qinwan GONG, Shenzhen (CN)

(73) Assignee: Shenzhen Guanke Technologies Co., Ltd., Shenzhen (CN)

(21) Appl. No.: 18/180,289

(22) Filed: Mar. 8, 2023

(30) Foreign Application Priority Data
Nov. 8, 2022 (CN) 202222973271.8

(51) Int. Cl. A61L 9/20; F21S 8/02; F21V 33/00

(52) U.S. Cl. CPC A61L 9/20; F21S 8/026; F21V 33/0096; A61L 2209/12

(57) ABSTRACT

A sterilizing panel lamp, including at least one housing, at least one frame body, at least one sterilization assembly, and at least one fan, where the frame body is arranged at the bottom peripheral side of the housing, the frame body encloses a mounting surface, at least an inlet grille, a lighting assembly, and an outlet grille are arranged along the mounting surface in a transverse or longitudinal direction, the inlet grille is detachably arranged between the frame body and lighting assembly, and the housing, frame body, inlet grille, lighting assembly and outlet grille enclose to form an air flow passage; the sterilization assembly is provided in the air flow passage; the fan is provided in the air flow passage, and the fan rotates to accelerate the speed at which air enters from the inlet grille and blows out of the outlet grille through the air flow passage.

### TECHNICAL FIELD

The present disclosure relates to the technical field of sterilizing equipment, and particularly relates to a sterilizing panel lamp.

### BACKGROUND

In the prior art, a panel lamp is usually installed at the top of a room (such as a ceiling), and in the installation mode, there are usually two types, namely, a hanging type and a built-in type, wherein the hanging type is to suspend the panel lamp below the ceiling via a hanging rope, and the built-in type is to integrally place the panel lamp inside the ceiling and the top plate, and only the bottom illumination part is leaked out, so as to have a better illumination effect and decorative effect. With the progress of the times, such panel lamps also have an air sterilization function, namely, an ultraviolet lamp is provided inside the panel lamp, air is sucked into the interior of the panel lamp through a fan, and after ultraviolet sterilization, air is discharged to complete sterilization.

Current sterilizing panel lamps typically have air inlet and outlet locations on the sides of the panel lamp, and such designs do not hide most of the structure of the panel lamp from the ceiling during installation and are particularly abrupt. Furthermore, when maintenance or replacement of the sterilization assembly inside the panel lamp is required, the frame of the panel lamp must also be removed, i.e. the handling of the sterilization assembly is inconvenient.

The foregoing is provided merely to aid in the understanding of the technical solution of the present application and is not an admission that the foregoing is prior art.

### SUMMARY

An object of the present disclosure is to provide a sterilizing panel lamp, aiming to improve the disassembly and assembly convenience of a germicidal module.

In order to achieve the above-mentioned object, the present disclosure proposes a sterilizing panel lamp, including:
at least one housing;
at least one frame body, wherein the frame body is arranged at the bottom peripheral side of the housing, and the frame body encloses a mounting surface, an inlet grille, a lighting assembly and an outlet grille are arranged in sequence along the transverse direction or the longitudinal direction of the mounting surface, the inlet grille is detachably arranged between the frame body and the lighting assembly, and the housing, the frame body, the inlet grille, the lighting assembly and the outlet grille enclose to form an air flow passage;
at least one sterilization assembly is provided in the air flow passage; and
at least one fan, the fan being disposed in the air flow path, the fan rotating to increase the speed at which air enters from the inlet grille and blows out of the outlet grille through the air flow path.

Other features and corresponding advantages of the present application are set forth in a later portion of the specification.

The problem solving idea and the relevant product design scheme of the present application are: at least one frame body is provided on a peripheral side of the bottom of the housing, the frame body encloses to form a mounting surface, and at least one inlet grille, at least one lighting assembly and at least one outlet grille are arranged in sequence along the transverse direction or the longitudinal direction of the mounting surface, so as to form an air flow passage enclosed by the housing, the frame body, the inlet grille, the lighting assembly and the outlet grille, and the arrangement of the inlet grille and the outlet grille can have a flow guiding effect on the air flow and improve the air flow circulation efficiency in the air flow passage. In addition, at least one sterilization assembly and at least one fan are provided in the air flow passage, and the air flow can enter the air flow passage from the inlet grille driven by the fan, then pass through the sterilization assembly to perform sterilization and disinfection, and then flow out from the outlet grille, thereby achieving the sterilizing function of the panel lamp. Furthermore, the inlet grille, the lighting assembly and the outlet grille of the sterilizing panel lamp are all arranged at the bottom of the housing, so that the bottom of the shell only needs to be exposed when the sterilizing panel lamp is installed, so that most of the structure thereof can be hidden inside the ceiling, so as to realize an concealed installation mode, and have a better lighting effect, ventilation effect and decorative effect. Wherein the inlet grille is detachably arranged between the frame body and the lighting assembly, and by detaching the inlet grille at the bottom of the sterilizing panel lamp, maintenance or replacement of the sterilization assembly arranged in the air flow passage can be performed, without disassembling the sterilizing panel lamp and disassembling the shell structure thereof, thereby improving the disassembly and assembly convenience of the germicidal module, which is beneficial to saving the labor cost of the operator.

### BRIEF DESCRIPTION OF THE DRAWINGS

FIG. 1 is a schematic structural diagram of an embodiment of a sterilizing panel lamp of the present disclosure;

FIG. 2 is a schematic view showing another perspective of the sterilizing panel lamp of FIG. 1;

FIG. 3 is an exploded view of the sterilizing panel lamp of FIG. 1;

FIG. 4 is a cross-sectional view of the sterilizing panel lamp of FIG. 1;

FIG. 5 is an enlarged partial view at A in FIG. 4;

FIG. 6 is a schematic view of the structure of the inlet grille and sterilization assembly of FIG. 1;

FIG. 7 is an exploded view of the intake grill and sterilization assembly of FIG. 5;

FIG. 8 is a partial structural view of the sterilizing panel lamp of FIG. 1;

FIG. 9 is a schematic structural diagram of another embodiment of the sterilizing panel lamp of the present disclosure;

FIG. 10 is a schematic view showing the structure of another view of the sterilizing panel lamp of FIG. 9.

The shape, size, proportion or positional relationship of the component parts of the product shown in the drawings can be actual data of the embodiments and fall within the scope of protection of the present application.

### DETAILED DESCRIPTION

The In order that the objects, aspects and advantages of the present application may be more clearly understood, embodiments of the present application will be described in further detail below with reference to the accompanying drawings. It should be understood that the particular embodiments described herein are illustrative only and are not restrictive.

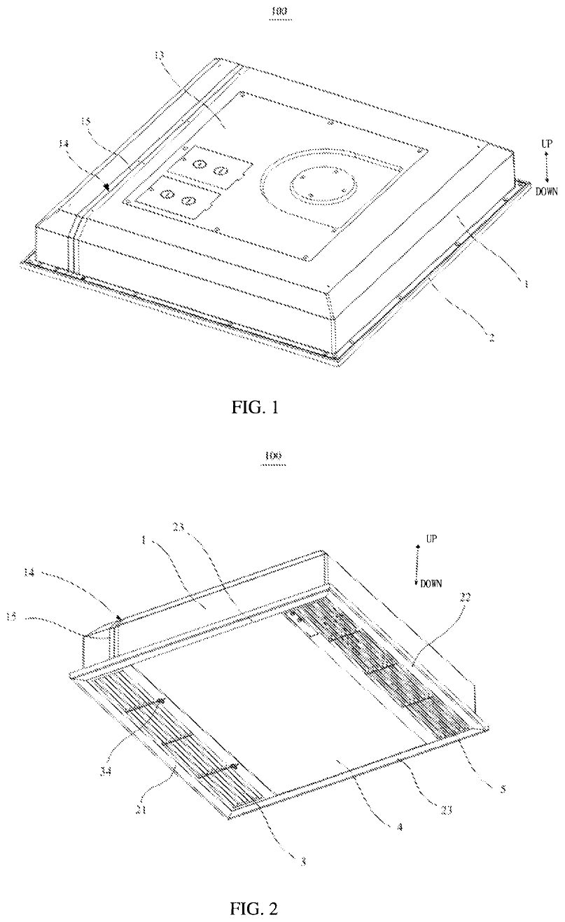

With reference to FIGS. 1 to 8, the present disclosure proposes a sterilizing panel lamp 100. In one embodiment of the present disclosure, the sterilizing panel lamp 100 comprises at least one shell 1, at least one frame body 2, at least one sterilization assembly 6 and at least one fan 7, wherein the frame body 2 is arranged at the bottom peripheral side of the shell 1, and the frame body 2 encloses a mounting surface 24, at least one inlet grille 3, at least one lighting assembly 4 and at least one outlet grille 5 are sequentially arranged along the transverse or longitudinal direction of the mounting surface 24, the inlet grille 3 is detachably arranged between the frame body 2 and the lighting assembly 4, and the shell 1, the frame body 2, the inlet grille 3, the lighting assembly 4 and the outlet grille 5 enclose to form an air flow passage B; the sterilization assembly 6 is provided in the air flow passage B; at least one fan 7 is provided in the air flow passage B, and the fan 7 rotates to increase the speed at which air enters from the inlet grille 3 and blows out of the outlet grille 5 through the air flow passage B.

The shell 1 serves as the main body structure of the sterilizing panel lamp 100, and is used to provide an installation space for the fan 7 and other structures of the sterilizing panel lamp 100, and can provide isolation and protection for components located in the shell 1. At the same time, the bottom of the shell 1 is provided with at least one frame body 2, and the frame body 2 is circumferentially arranged around the bottom of the shell 1 so as to form a mounting surface 24 for providing a mounting position for structures such as the inlet grille 3, the alighting assembly 4 and the outlet grille 5 so as to facilitate the installation of functional components at the bottom of the sterilizing panel lamp 100. By arranging the inlet grille 3, the lighting assembly 4 and the outlet grille 5 at the bottom of the sterilizing panel lamp 100, the built-in installation mode of the sterilizing panel lamp 100 is facilitated and the decorative effect of the sterilizing panel lamp 100 is improved. Specifically, the inlet grille 3, the lighting assembly 4 and the outlet grille 5 can be spliced and fixed on the inner side of the frame body 2 in sequence along the transverse or longitudinal direction of the mounting surface 24 so as to form a panel structure covering the bottom of the shell 1, and the air flow passage B can be formed by enclosing the shell 1, the frame body 2, the inlet grille 3, the lighting assembly 4 and the outlet grille 5 so as to improve the compactness of the structure at the mounting surface 24. Of course, several fixing plates can also be provided at the mounting surface 24 so as to separate several avoiding mounting openings from the inner side of the frame so as to enable the inlet grille 3 and the lighting assembly 4, the air outlet grille 5 is successively installed at the avoiding installation opening, and can be specifically and adaptively set according to needs. Wherein the inlet grille is provided in a detachable manner, and the inner side of the inlet grille 3 is provided with at least one sterilization assembly 6, so that the sterilizing panel lamp 100 does not need to be detached from the top of the room, and the shell 1 of the sterilizing panel lamp 100 also does not need to be detached and detached, and the sterilization assembly 6 located in the air flow passage B can be directly replaced or maintained by detaching the inlet grille 3, thereby improving the ease of detachment and detachment of the sterilization assembly 6 and saving the labor cost of the operator. Meanwhile, in order to further improve the ease of assembly and disassembly of the sterilization assembly 6, the sterilization assembly 6 may be provided on the inner surface of the inlet grille 3 in the following embodiments, so as to take out the sterilization assembly 6 in conjunction with the disassembly of the inlet grille 3, and so that the sterilization assembly 6 is provided on the inner surface of the inlet grille 3, so that the air flow entering the air flow passage B through the inlet grille 3 can sufficiently react with the sterilization assembly 6 to sterilize and disinfect the sterilizing panel lamp 100, thereby improving the sterilizing effect of the sterilizing panel lamp 100.

With reference to FIGS. 2 and 4 in combination, in one embodiment of the present disclosure, it is defined that the part of the frame body 2 contacting only the inlet grille 3 is a first frame plate 21, the inlet grille 3 comprises a first connection portion 31, a second connection portion 32 and a grille portion 33 located between the first connection portion 31 and the second connection portion 32, and the first connection portion 31 is rotatably inserted into the first frame plate 21; wherein the second connection portion 32 is fixed in cooperation with the lighting assembly 4 via at least one locking and releasing structure 34.

In the present embodiment, the inlet grille 3 includes a first connection portion 31, a second connection portion 32 for connecting the frame body 2 and the lighting assembly 4, respectively, and the grille portion 33 between the first connection portion 31 and the second connection portion 32. Wherein the part of the frame body 2 connected to the first connection portion 31 is the first frame plate 21, the first frame plate 21 is arranged substantially parallel to the first connection portion 31 and is located at the side of the inlet grille 3 which away from the lighting assembly 4, and the first connection portion 31 and the first frame plate 21 are rotatably inserted and matched; the grille portion 33 comprises several grills arranged at intervals and used for guiding the air flow so as to improve the air circulation efficiency; the second connection portion 32 and the lighting assembly 4 are arranged substantially in parallel, and can be locked and engaged with the lighting assembly 4 via at least one locking and releasing structure 34, so that the two ends of the inlet grille 3 can be limitedly fixed with the frame body 2 and the lighting assembly 4 respectively, so that the inlet grille 3 can be detachably arranged between the frame body 2 and the lighting assembly 4. Specifically, when it is necessary to disassemble the inlet grille 3, the second connection portion 32 and the lighting assembly 4 are firstly unlocked, so that the second connection portion 32 can rotate outwards as a free end of the inlet grille 3, so that the inlet grille 3 is inclined with respect to the mounting surface 24, at this time, the first connection portion 31 inserted into the first frame plate 21 can rotate with respect to the first frame plate 21, and when the first connection portion 31 rotates outwards to a certain angle with respect to the first frame plate 21, the first connection portion 31 and the first frame plate 21 can be disengaged by an outward force, so as to complete the disassembly process of the inlet grille 3. Similarly, when it is required to assemble the inlet grille 3, the first connection portion 31 is inserted into the first frame plate 21 at an inclined angle so as to achieve initial positioning of the inlet grille 3, and then the inlet grille 3 can be rotated towards the mounting surface 24 by an inward force until the second connection portion 32, which is the free end of the inlet grille 3, contacts the lighting assembly 4, and then the second connection portion 32 and the lighting assembly 4 can be cooperatively fixed by at least one locking and releasing structure 34, so that both ends of the inlet grille 3 are limitedly fixed, so as to complete the assembly process of the inlet grille 3.

Of course, the embodiments of the present invention are not limited thereto. In one embodiment of the present disclosure, a fixing plate substantially parallel to the first frame plate 21 is provided between the inlet grille 3 and the lighting assembly 4, and the second connection portion 32 is fixed in cooperation with the fixing plate via at least one locking and releasing structure 34.

In the present embodiment, the inlet grille 3, the lighting assembly 4 and the outlet grille 5 are sequentially arranged along the transverse direction or the longitudinal direction of the mounting surface 24, at least one fixing plate is further provided between the inlet grille 3 and the lighting assembly 4, and two sides of the fixing plate can respectively contact the inlet grille 3 and the lighting assembly 4 for providing a mounting basis for the inlet grille 3 and the lighting assembly 4. In particular, the fixing plate and the first frame plate 21 are parallel to each other for forming a relatively regular mounting space between the fixing plate and the first frame plate 21 to fit into the profile of the grille 3. The first connection portion 31 of the inlet grille 3 and the first frame plate 21 are rotatably inserted, and the second connection portion 32 can be locked with the fixed plate via at least one locking and releasing structure 34, so that the two ends of the inlet grille 3 are respectively limited and fixed. Likewise, when the second connection portion 32 and the fixing plate are unlocked, the second connection portion 32 can rotate outwards as the free end of the inlet grille 3, so that the inlet grille 3 is inclined with respect to the mounting surface 24, at this moment, the first connection portion 31 inserted into the first frame plate 21 can rotate with respect to the first frame plate 21, and when the first connection portion 31 rotates outwards to a certain angle with respect to the first frame plate 21, the first connection portion 31 and the first frame plate 21 can be disengaged by an outward acting force, so as to complete the process of disassembling the inlet grille 3; when it is required to assemble the inlet grille 3, the first connection portion 31 is inserted into the first frame plate 21 at an inclined angle so as to achieve initial positioning of the inlet grille 3, and then the inlet grille 3 can be rotated towards the mounting surface 24 by an inward force until the second connection portion 32, which is the free end of the inlet grille 3, contacts the fixing plate, and then the second connection portion 32 and the fixing plate can be fixed in cooperation by at least one locking and releasing structure 34, so that both ends of the inlet grille 3 are fixed at a limiting position, so as to complete the assembly process of the inlet grille 3.

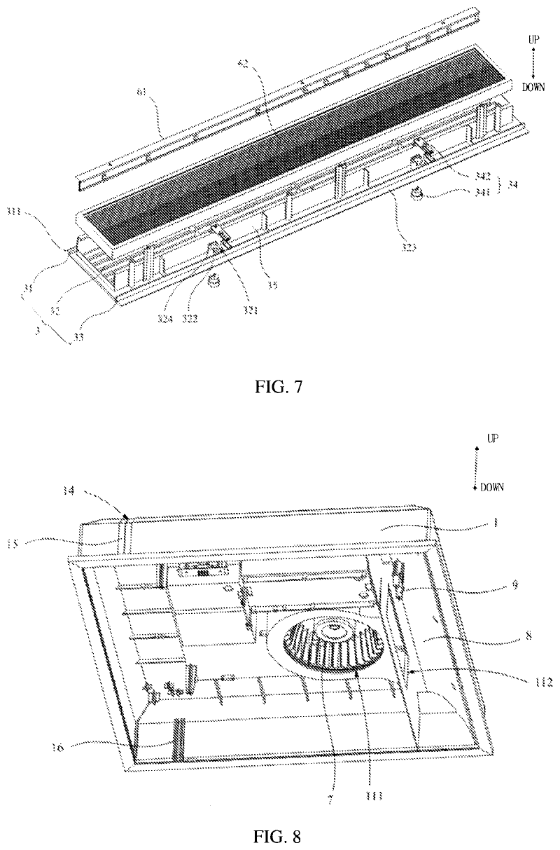

With reference to FIGS. 6 and 7 in combination, in one embodiment of the present disclosure, it is defined that the sterilizing panel lamp 100 has an up-and-down direction, and the second connection portion 32 is provided with at least one through-hole 321; the locking and releasing structure 34 comprises at least one rotating member 341 and at least one limiting member 342, wherein the rotating member 341 passes through the through-hole 321 and is exposed on the lower surface of the second connection portion 32, and the rotating member 341 can rotate along the central axis of the through-hole 321; one end of the limiting member 342 is fixed to the rotating member 341, and the other end extends from the rotating member 341 to the side of the rotating member 341 so as to partially protrude to the upper surface of the lighting assembly 4 as the rotating member 341 rotates, or to retract above the second connection portion 32 as the rotating member 341 rotates.

In the present embodiment, the second connection portion 32 is provided with at least one through-hole 321, and the through-hole 321 is provided through in the up-and-down direction for installing the locking and releasing structure 34, and the locking and releasing structure 34 can lock or release the second connection portion 32 to realize the detachable arrangement of the inlet grille 3. The locking and releasing structure 34 comprises at least one rotating member 341 and at least one limiting member 342, wherein the rotating member 341 passes through the through-hole 321 and can rotate along the central axis of the through-hole 321, and one end of the limiting member 342 is fixedly provided to the rotating member 341, and the other end extends from the rotating member 341 to the side of the rotating member 341. Rotating the rotating member 341 along the central axis of the through-hole 321 by an external force, that is to say, driving the limiting piece 342 to rotate synchronously and partially protrude to the upper surface of the lighting assembly 4, so that the limiting piece 342 is supported on the inner surface of the lighting assembly 4, and thus the second connection portion 32 can be stopped from rotating outwards, so that the second connection portion 32 is locked to the mounting surface 24; however, when the rotating member 341 rotates in the opposite direction along the central axis of the through-hole 321, the limiting member 342 is driven to rotate synchronously and retract above the second connection portion 32, so that the second connection portion 32 is no longer supported by the lighting assembly 4, and the restriction of the inlet grille by the lighting assembly 4 can be released. Wherein the inner wall of the through-hole 321 is provided in stepped shape, and the aperture of the end of the through-hole 321 close to the outer surface of the first connection portion 31 is greater than the aperture of the end thereof away from the outer surface of the first connection portion 31, so as to facilitate the installation and fixing of the rotating member 341 from the outside to the inside; the stopper 342 is provided on the inner surface of the rotating member 341 and is detachably coupled with the rotating member 341 by a screw so as to facilitate the assembly of the locking structure 34.

Further, in order to facilitate the user to control the rotating member 341 to rotate, the rotating member 341 is exposed on the lower surface of the second connection portion 32, and the lower surface of the rotating member 341 is provided with a knob structure, so that the user can rotate the knob structure via a hexagonal wrench so as to rotate the rotating member 341 and drive the limiting piece 342 to rotate so as to lock or release the second connection portion 32.

With reference to FIGS. 6 and 7 in combination, in one embodiment of the present disclosure, at least one boss 322 is provided on the upper surface of the second connection portion 32, the top surface of the boss 322 is arranged higher than the upper surface of the second connection portion 32, at least one through-hole 321 is provided on the top surface of the boss 322, and the rotating member 341 passes through the through-hole 321 and is exposed to the lower surface of the second connection portion 32.

In the present embodiment, the inner surface of the second connection portion 32 is provided with a boss 322 for mounting the locking and releasing structure 34, the upper surface of the boss 322 is arranged higher than the upper surface of the second connection portion 32, the top surface of the boss 322 is provided with a through-hole 321, and the rotating member 341 passes through the through-hole 321 and is exposed to the lower surface of the second connection portion 32. The arrangement of the boss 322 can, on the one hand, improve the installation foundation for the rotating member 341 and improve the structural strength of the second connection portion 32, so as to improve the positional stability of the rotating member 341, and on the other hand, the upper surface of the boss 322 can raise the height position of the limiting member 342, so as to facilitate the limiting member 342 to be rotationally protruded to the side, and prevent the limiting member 342 from interfering with other structures provided on the installation surface 24, such as the lighting assembly 4, during rotation.

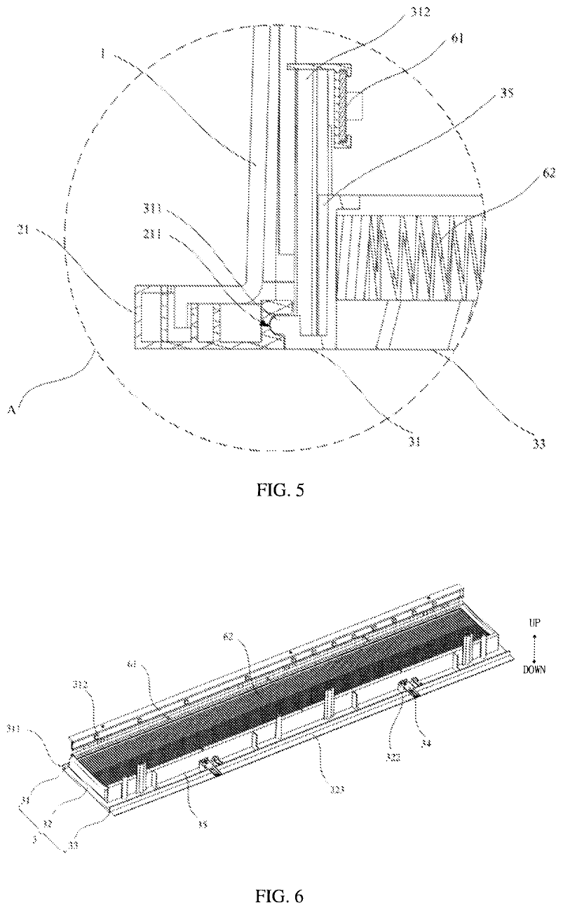

With reference to FIGS. 5 and 6 in combination, in one embodiment of the present disclosure, the second connection portion 32 comprises at least one plate body 323 extending away from the first connection portion 31, the upper surface of the plate body 323 is lower than the upper surface of the second connection portion 32, and when the limiting portion protrudes to the upper surface of the lighting assembly 4, the upper surface of the plate body 323 abuts the lower surface of the lighting assembly 4.

In this embodiment, the side of the second connection portion 32 remote from the first connection portion 31 is provided with at least one plate body 323 protruding therefrom, and the plate body 323 is provided to extend in the direction in which the inlet grille 3, the lighting assembly 4, and the outlet grille 5 are arranged so as to protrude below the lighting assembly 4, thereby serving as an assembly platform between the lighting assembly 4 and the inlet grille 3. When the inlet grille 3 is locked at the mounting surface 24, a part of the locking and releasing structure 34 provided on the second connection portion 32 protrudes from the upper surface of the lighting assembly 4, and the upper surface of the plate body 323 of the second connection portion 32 abuts against the lower surface of the lighting assembly 4. At this time, the locking and releasing structure 34 and the second connection portion 32 can be respectively provided on the upper and lower surfaces of the lighting assembly 4, so as to prevent the second connection portion 32 from being further displaced inwardly or outwardly, so that the inlet grille 3 and the lighting assembly 4 are firmly fitted and fixed, and the position stability of the second connection portion 32 is improved.

With reference to FIGS. 4 and 5 in combination, in one embodiment of the present disclosure, the first frame plate 21 is concavely provided with at least one groove 211 towards one side of the inlet grille 3, the cross section of the groove 211 is arc-shaped, the first connection portion 31 extends out of the convex strip 311 in the direction of the groove 211, and the convex strip 311 is rotatably inserted into the groove 211;

In the present embodiment, the first frame plate 21 is concavely provided with the groove 211 toward one side of the inlet grille 3, the groove 211 is oppositely arranged along the length direction of the first frame plate 21, and the first connection portion 31 projects in the direction of the groove 211 with at least one convex strip 311 having a shape adapted to the groove 211. The first connection portion 31 and the frame body 2 can be fixedly connected by a plug-fit of the convex strip 311 and the groove 211. In addition, the groove 211 and the convex strip 311 are provided in a circular arc shape in cross section, so that the first connection portion 31 is rotatably inserted into the first frame plate 21, so that the inlet grille 3 after the second connection portion 32 is unlocked can be rotated outwards and separated from the first frame plate 21. Among other things, the rounded configuration also helps to reduce friction between the convex strip 311 and the groove wall of the groove 211 to facilitate a rotational fit between the first connection 31 and the first frame plate 21.

With reference to FIGS. 4 and 7 in combination, in one embodiment of the present disclosure, the grille portion 33 comprises a plurality of grills, the grills are connected in staggered manner at intervals to form grille holes, and the grills are obliquely arranged below towards the first frame plate 21.

In this embodiment, a plurality of grills are alternately connected to form grille holes through which air flows into the air flow passage B. Wherein the grille bars are obliquely arranged towards the first frame plate 21 in the air inlet direction, so that the outer wall of the grille bars can have a guiding effect on the air flow so as to guide the external air flow quickly into the air flow passage B, thereby helping to improve the ventilation rate of the sterilizing panel lamp 100, so as to improve the killing efficiency of the sterilizing panel lamp 100.

Referring to FIGS. 6 and 7 in combination, in one embodiment of the present disclosure, two mutually perpendicular stoppers 324 protruding upward from the upper surface of the second connection portion 32 are provided on the rotation path of the limiting member 342 to rotate the limiting member 342 between 0-90 degrees.

In the present embodiment, the stopper 342 of the lock and release structure 34 is provided on the upper surface of the second connection portion 32, and the stopper 342 can rotate around the rotation axis of the rotating member 341 driven by the rotating member 341. At this time, by providing two stoppers 324 protruding from the upper surface of the second connection portion 32 and stopping the two stoppers 324 on the rotation path of the stopper 342, the length range of the rotation path of the stopper 342 can be limited, and excessive displacement of the stopper 342 can be prevented from interfering with other structures of the sterilizing panel lamp 100. Specifically, the two stops 324 are arranged perpendicular to each other so as to form a right angle between the two stops 324, and the limiting member 342 is provided between the two stops 324 and can rotate back and forth between 0 degrees and 90 degrees, and when the limiting member 342 abuts one of the stops 324, the limiting member 342 protrudes from the upper surface of the lighting assembly 4, and when the limiting member 342 abuts the other stop 324, the limiting member 342 retracts above the second connection portion 32 and is substantially parallel to the second connection portion 32.

With reference to FIGS. 4 to 7 in combination, in one embodiment of the present disclosure, the first connection portion 31 of the inlet grille 3 is provided with at least one bracket 312, and the bracket 312 extends towards the top surface of the shell 1; the sterilization assembly 6 includes at least one ultraviolet lamp panel 61 provided on a side of the bracket 312 facing the grille portion 33.

In the present embodiment, the upper surface of the first connection portion 31 of the inlet grille 3 is provided with at least one bracket 312, which extends toward the top surface of the shell 1 in the height direction of the inlet grille 3 and serves as mounting structure for the sterilization assembly 6 to support and fix the sterilization assembly 6. The sterilization assembly 6 comprises at least one ultraviolet lamp plate 61, wherein the ultraviolet lamp plate 61 is provided on one side of the bracket 312 facing the grille portion 33, and the light-emitting surface of the ultraviolet lamp plate 61 is provided directly above the grille portion 33, and the ultraviolet ray having a sterilizing effect can sterilize the outside air entering the air flow passage B from the grille portion 33.

With reference to FIGS. 4 to 7 in combination, in one embodiment of the present disclosure, the first connection portion 31 and the second connection portion 32 of the inlet grille 3 relatively extend upwards out of the mounting plate 35, the sterilization assembly 6 comprises at least one air filter 62, the air filter 62 is inserted between the two mounting plates 35 and is adhered to the upper surface of the grille portion 33, and the upper ends of the two mounting plates 35 relatively project limiting strip to limit the upward movement of the air filter 62.

In the present embodiment, the upper surface of the first connection portion 31 and the upper surface of the second connection portion 32 of the inlet grille 3 respectively extend upward with mounting plates 35, which are disposed opposite to each other. The sterilizing unit 6 includes at least one air filter 62 interposed between the two mounting plates 35 so as to be attached to the upper surface of the grille portion 33, and when outside air enters the air flow passage B from the grille portion 33 by the fan 7, it is primarily purified and filtered through the air filter 62 positioned on the upper surface of the grille portion 33. Meanwhile, the upper end of each of the mounting plates 35 is protruded toward the other mounting plate 35 with a stopper bar, which is stopped above the air filter 62, for limiting the upward movement of the air filter 62, so that the air filter 62 is stably caught above the grille portion 33 without being deviated or slipped due to an external impact, thereby improving the positional stability of the air filter 62.

With reference to FIG. 2, in one embodiment of the present disclosure, it is defined that the part of the frame body 2 contacting only the outlet grille 5 is the second frame plate 22, and the remaining part of the frame body 2 is the third frame plate 23; the third frame plates 23 are two and the two third frame plates 23 are arranged in parallel, the third frame plates 23 abut against the edge of the lower surface of the lighting assembly 4 so as to support the lighting assembly 4, and the length of a connecting line between two ends of the inlet grille 3 close to the third frame plates 23 is slightly less than the distance between the two third frame plates 23; the outlet grille 5 is inserted into the second frame plate 22.

In the present embodiment, the end of the inlet grille 3 away from the lighting assembly 4 is in contact with the frame body 2, and the part of the frame body 2 in contact with the inlet grille 3 is the first frame plate 21 of the frame body 2; one end of the outlet grille 5 away from the lighting assembly 4 is in contact with the frame body 2, and the part of the frame body 2 in contact with the outlet grille 5 is the second frame plate 22 of the frame body 2; the frame body 2 further comprises two third frame plates 23, wherein the two third frame plates 23 are arranged parallel to each other, and two ends of the third frame plates 23 respectively abut against the first frame plate 21 and the second frame plate 22. The first frame plate 21, the second frame plate 22 and the two third frame plates 23 enclose a mounting surface 24 for providing a mounting base for the inlet grille 3, the lighting assembly 4 and the outlet grille 5. Specifically, each of the third frame plates 23 abuts one side edge of the lower surface of the lighting assembly 4 to support the lighting assembly 4 such that the lighting assembly 4 is embedded in central region of the mounting surface 24, and at this time, two sides of the lighting assembly 4 are each formed with a avoidance region for installing the inlet grille 3 and the outlet grille 5. One end of the outlet grille 5 is inserted into the second frame plate 22 so as to facilitate the installation of the outlet grille 5; the length of the connection line between the two ends of the inlet grille 3 close to the third frame plate 23 is slightly less than the distance between the two third frame plates 23, so that the two ends of the inlet grille 3 do not interfere with the two third frame plates 23 when the inlet grille 3 is assembled and disassembled, thereby facilitating the detachable connection of the inlet grille 3.

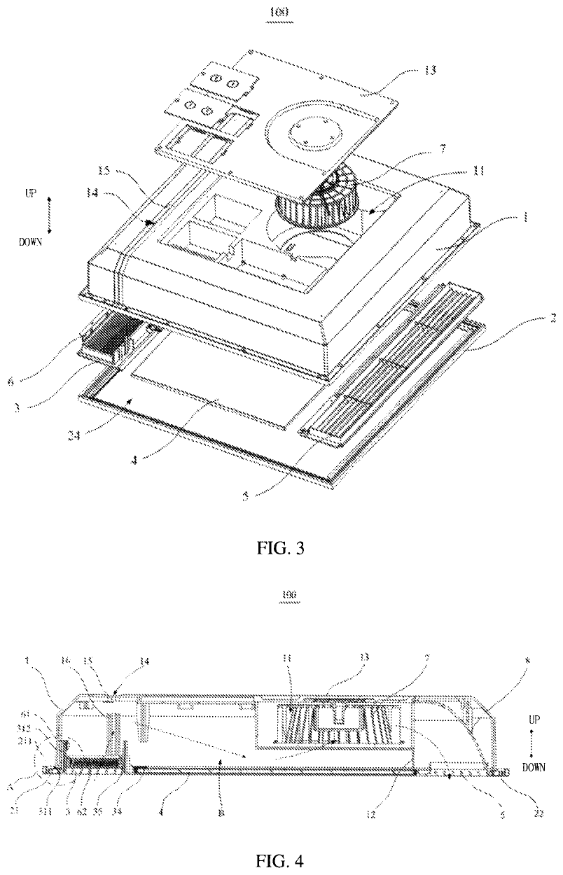

With reference to FIGS. 3 and 8 in combination, in one embodiment of the present disclosure, the shell 1 is recessed downwards at a position close to the outlet grille 5 to form an receiving cavity 11, and the fan 7 is provided in the receiving cavity 11; the fan 7 is arranged towards the lighting assembly 4, at least one air inlet 111 is provided at the bottom of the receiving cavity 11, at least one air outlet 112 is provided at the side of the receiving cavity 11 close to the outlet grille 5, and the fan 7 rotates so as to suck the air in the air flow passage B from the air inlet 111 and discharge the air from the air outlet 112 to the outlet grille 5 via the blades.

In this embodiment, the upper surface of the shell 1 at a position close to the outlet grille 5 is depressed downward to form a receiving cavity 11 for mounting the fan 7. At least one air inlet 111 is provided at the bottom of the receiving cavity 11 at the position corresponding to the lighting assembly 4, the fan 7 in the receiving cavity 11 is arranged towards the lighting assembly 4, and the receiving cavity 11 is provided with at least one air outlet 112 at the side close to the outlet grille 5, so that the air flow can firstly flow from the inlet grille 3 towards the upper side of the lighting assembly 4 under the driving of the fan 7, and is sucked into the receiving cavity 11 at the upper side of the lighting assembly 4 via the air inlet 111, then is discharged into the upper side of the outlet grille 5 via the blades of the fan 7 from the air outlet 112 of the receiving cavity 11, and finally is conducted out to the outside via the outlet grille 5. Wherein the fan 7 is arranged towards the lighting assembly 4, enabling the airflow to quickly flow through the space above the lighting assembly 4, and taking away the heat emitted by the lighting assembly 4 in time, so that the heat dissipation efficiency of the lighting assembly 4 can be improved, which is beneficial to extending the service life of the lighting assembly 4. Furthermore, the concave formation of the receiving cavity 11 on the top surface of the shell 1 is also advantageous in making full use of the headspace inside the shell 1, ensuring that the thickness of the structure of the shell 1 is not increased in a large size in the up and down direction, so as to achieve a compact structural configuration.

With reference to FIGS. 4 and 8, in one embodiment of the present disclosure, at least one partition plate 12 is provided between the upper surface of the lighting assembly 4 and the bottom of the receiving cavity 11 near one side of the outlet grille 5, so that the air entering the air flow passage B passes through the receiving cavity 11.

In the present embodiment, the partition plate 12 can block the direct flow of the air flow in the bottom space of the accommodating chamber 11, so as to facilitate the air entering the air flow passage B from one end of the sterilizing panel lamp 100, and quickly blowing out from the other end of the sterilizing panel lamp 100 to the external environment after all of the air passes through the accommodating chamber 11, so as to enable the air flow in the air flow passage B to flow in a unidirectional and smooth manner, and prevent the air flow in the bottom space of the accommodating chamber 11 from turning back to affect the flow and mobility of the air flow in the air flow passage B.

With reference to FIG. 3, in one embodiment of the present disclosure, at least one cover 13 is provided above the fan 7 and is fixed by means of at least one screw so as to seal the receiving cavity 11.

In the present embodiment, the upper surface of the shell 1 is recessed downward to form the receiving cavity 11, the fan 7 is provided in the receiving cavity 11, and a cover 13 is provided above the fan 7, the cover 13 is detachably covered at the recess by at least one screw to form the top wall of the receiving cavity 11, so that the fan 7 provided inside the receiving cavity 11 can be protected, and the fan 7 in the receiving cavity 11 can be maintained or replaced by detaching the cover 13, thereby improving the maintenance convenience of the fan 7.

Further, the fan 7 is suspended from the inner surface of the cover 13 so as to leave a gap between the fan 7 and the bottom of the accommodating chamber 11, so that the airflow can be rapidly circulated toward the inside of the accommodating chamber 11, which is advantageous in increasing the air circulation rate of the sterilizing panel lamp 100.

With reference to FIGS. 4 and 8 in combination, in one embodiment of the present disclosure, at least one arc-shaped air guiding plate 8 is provided on one side of the outlet grille 5 close to the second frame plate 22, and the arc-shaped air guiding plate 8 extends from the outlet grille 5 towards the top of the air outlet 112.

In this embodiment, at least one arc-shaped air guiding plate 8 is provided on the side of the air outlet grille 5 close to the second frame plate 22, and the arc-shaped air guiding surface 8 extends from the air outlet grille 5 to the upper side of the air outlet 112 for guiding the air flow flowing out from the air outlet 112 to the air outlet grille 5, so that the air flow rapidly flows to the external environment through the air outlet grille 5, thereby increasing the air circulation rate of the air flow passage B. Further, in order to improve the positional stability of the arc-shaped wind deflector 8, the top surface of the arc-shaped wind deflector 8 may be further provided with at least one convex strip structure extending upward from the top surface of the arc-shaped wind deflector 8 and abutting against the inner surface of the top wall of the shell 1, for supporting and fixing the arc-shaped wind deflector 8.

With reference to FIG. 7, in one embodiment of the present disclosure, at least one negative ion air purifier 9 is provided above the outlet grille 5 corresponding to the air outlet 112.

In the present embodiment, the receiving cavity 11 is provided with at least one air outlet 112 at the side close to the outlet grille 5, and the air flow in the receiving cavity 11 flows towards the upper side of the outlet grille 5 via the air outlet 112, and then is discharged into the external environment via a plurality of grid holes of the outlet grille 5. By providing the negative ion air purifier 9 above the air outlet grille 5 and providing the negative ion air purifier 9 corresponding to the air outlet 112, the air flow flowing from the air outlet 112 toward the air outlet grille 5 can be sufficiently purified by the negative ion air purifier 9, so as to improve the air purification effect of the sterilizing panel lamp 100.

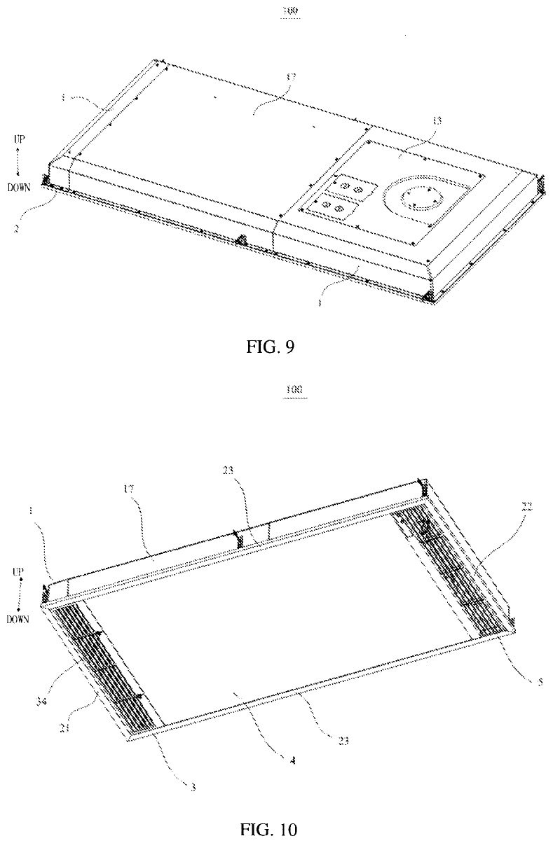

With reference to FIG. 1 or FIG. 9, in one embodiment of the present disclosure, it is defined that the direction in which the inlet grille 3, the lighting assembly 4 and the outlet grille 5 are sequentially arranged is the length direction, and the shell 1 is provided with at least one pre-cut line 14 along the width direction.

In the present embodiment, the shell 1 is provided with at least one pre-cut line 14 along the width direction, and the shell 1 can perform a further cutting operation along the pre-cut line 14 to divide the shell 1 into two parts along the width direction, and then two ends of the pre-preconfigured packaging sheet 17 can be respectively connected with the two parts of the shell 1 to be assembled into a shell 1 assembly having larger size in the length direction, so that the area of the mounting face 24 of the sterilizing panel lamp 100 can be increased, thereby enabling to replace the lighting assemblies 4 of different sizes or to increase the number of assemblies of the lighting assemblies 4 of the original size by applicability at the mounting face 24. The sterilizing panel lamp 100 is adapted to different usage requirements. Wherein the pre-cut line 14 can be a groove structure provided on the outer surface of the shell 1, and the groove structure can reduce the wall thickness of the shell 1 so as to cut and separate the shell 1; the sectional shape of the packaging sheet 17 in the width direction is substantially the same as the sectional shape of the shell 1 to maintain the uniform appearance of the sterilizing panel lamp 100.

With reference to FIGS. 1 to 10, in one embodiment of the present disclosure, the pre-cut line 14 is provided near one end of the inlet grille 3 and passes through the outer surface of the shell 1; the connection face 15 is provided along two sides of the pre-cut line 14, and the outer surface of the connection face 15 is provided slightly lower than the outer surface of the shell 1 for fixedly connecting two ends of the pre-preconfigured encapsulation sheet 17 to the two connection faces 15 respectively after cutting and separating along the pre-cut line 14; the inner surface of the shell 1 is provided with reinforcing convex ribs 16 on both sides of the pre-cut line 14.

In the present embodiment, the pre-cut line 14 is provided at the end of the shell 1 near the inlet grille 3, and the pre-cut line 14 passes through the upper surface of the shell 1 and both sides connected to the two third frames 2. Furthermore, two connecting faces 15 are provided on both sides of the pre-cut line 14, the outer surfaces of the two connecting faces 15 are provided slightly lower than the upper surface of the shell 1 so as to form a step structure on both sides of the cut line, and two ends of the pre-preconfigured packaging sheet 17 are respectively pressed on the two connecting faces 15 and can be locked and fixed with the two connecting faces 15 by means of screws. By providing the connection surface 15, the mounting and positioning of the encapsulation sheet 17 can be facilitated, and the outer surface of the encapsulation sheet 17 can be made flush with the upper surface of the shell 1 to ensure the flatness of the upper surface of the sterilizing panel lamp 100. In addition, the inner surface of the shell 1 is further provided with reinforcing convex ribs 16, and the reinforcing convex ribs 16 are provided with a plurality of reinforcing convex ribs and are arranged in pairs on both sides of the pre-cut line 14, so as to improve the structural strength of the portions of the shell 1 on both sides of the pre-cut line 14 so as to facilitate the subsequent cutting processing of the shell 1.

The above is merely a preferred embodiment of the present disclosure, and thus does not limit the scope of the patent of the present disclosure. Any equivalent structural transformation made by using the contents of the description and the drawings of the present disclosure under the concept of the present disclosure, or direct/indirect application in other relevant technical fields, is included in the scope of the present disclosure.

What is claimed is:

1. A sterilizing panel lamp comprising:
at least one housing;
at least one frame body, wherein the frame body is arranged at the bottom peripheral side of the housing, the frame body encloses to form a mounting surface, an inlet grille, a lighting assembly and an outlet grille are arranged in sequence along the transverse direction or the longitudinal direction of the mounting surface, the inlet grille is detachably arranged between the frame body and the lighting assembly, and the housing, the frame body, the inlet grille, the lighting assembly and the outlet grille enclose to form an air flow passage;
at least one sterilization assembly is provided in the air flow passage; and
at least one fan disposed in the air flow path, the fan rotating to increase the speed at which air enters from the inlet grille and blows out of the outlet grille through the air flow path.

2. The sterilizing panel lamp as claimed in claim 1, wherein the portion of the frame body defined to contact only the inlet grille is the first frame plate, the inlet grille comprises a first connection portion, a second connection portion, and a grille portion between the first connection portion and the second connection portion, the first connection portion rotatably inserted into the first frame plate;
wherein the second connection portion is fixed in cooperation with the lighting assembly via at least one locking and releasing structure; or a fixing plate substantially parallel to the first frame plate is provided between the inlet grille and the lighting assembly, and the second connection portion is fixed in cooperation with the fixing plate via at least one locking and releasing structure.

3. The sterilizing panel lamp as claimed in claim 2, wherein the sterilizing panel lamp is defined to have an up and down direction and the second connection portion is provided with at least one through-hole; and
the locking and releasing structure comprises:
at least one rotating member penetrating the through-hole and exposed to the lower surface of the second connection portion, the rotating member being rotatable along the central axis of the through-hole; and
at least one limiting member, wherein one end of the limiting member is fixed to the rotating member, and the other end extends from the rotating member to the side of the rotating member, so as to partially protrude to the upper surface of the lighting assembly as the rotating member rotates or retract to above the second connection portion as the rotating member rotates.

4. The sterilizing panel lamp as claimed in claim 3, wherein the upper surface of the second connection portion is provided with at least one boss, the top surface of the boss is arranged higher than the upper surface of the second connection portion, the top surface of the boss is provided with at least one through-hole, and the rotating member passes through the through-hole and is exposed to the lower surface of the second connection portion;
and/or, the second connection portion comprises at least one plate body extending away from the first connection portion, the upper surface of the plate body is lower than the upper surface of the second connection portion, and when the limiting part protrudes to the upper surface of the lighting assembly, the upper surface of the plate body abuts the lower surface of the lighting assembly;
and/or one side of the first frame plate facing the inlet grille is concavely provided with a groove, the cross section of the groove is in the shape of an arc, the first connection portion extends out of a convex strip in the direction of the groove, and the convex strip is rotatably inserted into the groove;
and/or the grille portion comprises a plurality of grills, the grills are connected in a staggered manner at intervals to form grid holes, and the grills are obliquely arranged below towards the first frame plate;
and/or the upper surface of the second connection portion is provided with two mutually perpendicular stoppers protruding upwards, and the two stoppers are provided on the rotation path of the limiting member, so that the limiting member rotates between 0-90 degrees.

5. The sterilizing panel lamp as claimed in claim 2, wherein the first connection portion of the inlet grille is provided with at least one bracket extending toward the top surface of the housing, the sterilization assembly comprising at least one ultraviolet lamp panel disposed on a side of the bracket facing the grille portion;
and/or the first connection portion and the second connection portion of the inlet grille extend relatively upward out of the mounting plate, the sterilization assembly comprises at least one air filter, the air filter is inserted between the two mounting plates and is adhered to the upper surface of the grille portion, and the upper ends of the two mounting plates protrude relatively from the limiting strip so as to limit the upward movement of the air filter.

6. The sterilizing panel lamp as claimed in claim 2, wherein the portion of the frame that is defined to contact only the outlet grille is the second frame plate and the remaining portion of the frame is the third frame plate;
the third frame plates are two in number and the two third frame plates are arranged in parallel, the third frame plates abut an edge of the lower surface of the lighting assembly to support the lighting assembly, and the length of a connecting line between two ends of the inlet grille close to the third frame plates is slightly less than the distance between the two third frame plates;
the outlet grille is plugged to the second frame plate.

7. The sterilizing panel lamp as claimed in claim 6, wherein the upper surface of the shell is recessed downwardly at a position adjacent to the outlet grille to form a receiving cavity, the fan being disposed within the receiving cavity;
the fan is arranged towards the lighting assembly, at least one air inlet is provided at the bottom of the receiving cavity, at least one air outlet is provided at the side of the receiving cavity close to the outlet grille, and the fan rotates so as to suck air in the air flow passage from the air inlet and discharge the air from the air outlet to the outlet grille via the blades of the fan.

8. The sterilizing panel lamp as claimed in claim 7, wherein at least one baffle is disposed between the upper surface of the lighting assembly and the bottom of the receiving cavity adjacent the side of the outlet grille to allow air entering the air flow passage to pass through the receiving cavity;
and/or at least one cover is provided above the fan and is fixed by a screw so as to seal the receiving cavity;
and/or the side of the outlet grille close to the second frame plate is provided with arc-shaped air guiding plate, and the arc-shaped air guiding plate extends from the outlet grille towards the upper part of the air outlet;
and/or at least one negative ion air purifier is provided above the outlet grille corresponding to the air outlet.

9. The sterilizing panel lamp as claimed in claim 1, wherein the direction in which the inlet grille, the lighting assembly and the outlet grille are sequentially arranged is defined as the length direction and the shell is provided with pre-cut lines in the width direction.

10. The sterilizing panel lamp as claimed in claim 9, wherein the pre-cut line is disposed proximate one end of the inlet grille and extends through outer surface of the housing;
a connecting surface is provided along two sides of the pre-cut line, and the outer surface of the connecting surface is provided slightly lower than the outer surface of the shell for fixedly connecting two ends of the pre-preconfigured packaging sheet to the two connecting surfaces respectively after cutting and separating along the pre-cut line;
the inner surface of the shell is provided with reinforcing convex ribs on both sides of the pre-cut line.
